(19) United States
(12) Patent Application Publication
(10) Pub. No.: US 2022/0062457 A1
(43) Pub. Date: Mar. 3, 2022

(54) ELECTROLYTIC HAND SANITIZER DEVICE

(71) Applicant: Rheem Manufacturing Company, Atlanta, GA (US)

(72) Inventors: Harsha Satyanarayana, Norwalk, CT (US); Christopher M. Haden, Shelton, CA (US)

(21) Appl. No.: 17/004,514

(22) Filed: Aug. 27, 2020

(51) Int. Cl. A61L 2/03; A61Q 19/10; A61L 2/00; A61L 2/24

(52) U.S. Cl. CPC A61L 2/035; A61Q 19/10; A61L 2202/11; A61L 2/24; A61L 2/0047

(57) ABSTRACT

A hand sanitizer device is disclosed. The hand sanitizer device can include one or more storage chambers configured to store at least one of water and salt. The hand sanitizer device can be configured to receive a request for an amount of electrolyzed saltwater product for performing a hand disinfecting action. In response to receiving the request, the hand sanitizer device can automatically introduce a predetermined amount of water and a predetermined amount of salt into a reaction chamber and can be configured to provide a current of electricity from an electricity source and through the reaction chamber, thereby electrolyzing the contents in the reaction chamber to form an electrolyzed saltwater product.

### FIELD OF THE DISCLOSURE

The present disclosure relates generally to hand sanitizers and, in particular, to hand sanitizers produced on-demand through electrolytic processes and devices for making the same.

### BACKGROUND

Hand sanitation has become a vital part of everyday life. Generally, hand sanitizers are alcohol-based and are not safe if ingested. Thus, poisoning by ingestion hand sanitizer can be a problem, especially among children. Also, hand sanitizers are typically manufactured far from the location of end use, and any impediments to related logistics, supply chain, and/or demand can create a shortage. An additional challenge present with current hand sanitizers is that alcohol-based hand sanitizers can be flammable and pose a fire hazard, not only at the point of end use, but during transportation, which could cause severe damage to the supply chain.

As an alternative to alcohol-based hand sanitizers, hypochlorous acid (HOCl) has proven to be an effective disinfectant. Indeed, hypochlorous acid is listed on the Environmental Protective Agency's list of disinfectants for use against SARS-COV-2 (COVID-19). See United States Environmental Protective Agency, List N: Disinfectants for Use Against SARS-CoV-2 (COVID-19), EPA Reg. No. 93908-1, Jul. 16, 2020. Moreover, hypochlorous acid is easily manufacturable, such as by electrolyzing saltwater. However, a major disadvantage of hypochlorous acid is that it has a short shelf life of approximately four hours and is thus most effective when used quickly after production. Therefore, improving the production and dispensing of electrolyzed saltwater as hand sanitizers can improve the general public health and help to fight against not only COVID-19, but other pathogens that can be combatted with the use of hand sanitizer.

What is needed, therefore, are improved hand sanitizers and devices for the same that can produce electrolyzed saltwater quickly and efficiently. The present disclosure addresses this need as well as other needs that will become apparent upon reading the description below in conjunction with the drawings.

### BRIEF SUMMARY

The present disclosure relates generally to hand sanitizers and, in particular, to hand sanitizers produced on-demand through electrolytic processes and devices for making the same. The disclosed technology can provide a hand sanitizer device comprising: a first storage chamber, a second storage chamber, a reaction chamber connected to the first storage chamber and the second storage chamber, and an outlet connected to the reaction chamber. The reaction chamber can be connected to an electric potential configured to provide a current through the reaction chamber. The current can induce a voltage drop across the reaction chamber to electrolyze the contents in the reaction chamber and create a product. The first storage chamber can contain water and the second can contain a salt. The salt can be sodium chloride.

The hand sanitizer device can further comprise a holding tank connected to the reaction chamber and configured to store the product produced in the reaction chamber. The holding tank can also be connected to the outlet, and the holding tank can be configured to dispense a quantity of the product through the outlet when requested.

The hand sanitizer device can further comprise a vacuum, a pump, a piston, or other pressure differential-creating device configured to draw the product out of the outlet. The pressure differential-creating device can be contained within the reaction chamber or the holding tank. Alternatively, the pressure differential-creating device can be attached to an exterior portion of the hand sanitizer device.

The hand sanitizer device can further comprise a third storage chamber. The third storage chamber can be connected to the reaction chamber. The third storage chamber can contain an additive to be mixed into the hand sanitizer. The additive can be selected from the group consisting of: a moisturizer, a surfactant, a vinegar, an emulsifier, and a gel. The third storage chamber can be configured to mix the additive into the reaction chamber before the current is applied or after the current is applied but prior to dispensing the hand sanitizer.

These and other aspects of the present disclosure are described in the Detailed Description below and the accompanying figures. Other aspects and features of examples of the present disclosure will become apparent to those of ordinary skill in the art upon reviewing the following description of specific examples of the present disclosure in concert with the figures. While features of the present disclosure may be discussed relative to certain examples and figures, all examples of the present disclosure can include one or more of the features discussed herein. Further, while one or more examples may be discussed as having certain advantageous features, one or more of such features may also be used with the various examples of the disclosure discussed herein. In similar fashion, while examples may be discussed below as device, system, or method examples, it is to be understood that such examples can be implemented in various devices, systems, and methods of the present disclosure.

### BRIEF DESCRIPTION OF THE DRAWINGS

The accompanying drawings, which are incorporated in and constitute a part of this specification, illustrate multiple examples of the presently disclosed subject matter and serve to explain the principles of the presently disclosed subject matter. The drawings are not intended to limit the scope of the presently disclosed subject matter in any manner.

FIG. 1 illustrates a component diagram of an example hand sanitizer device, in accordance with the present disclosure.

FIG. 2 illustrates a component diagram of an example hand sanitizer device, in accordance with the present disclosure.

FIG. 3 illustrates a component diagram of an example hand sanitizer device, in accordance with the present disclosure.

FIG. 4 illustrates a flowchart of an example method for dispensing hand sanitizer, in accordance with the present disclosure.

### DETAILED DESCRIPTION

Throughout this disclosure, systems and methods are described with respect to hand sanitizer devices. The disclosed hand sanitizer devices can be configured to create and/or dispense an amount of a sanitizing agent in response to receiving a request for the sanitizing agent (e.g., detecting the presence of a user or a user's hand). In particular, the disclosed heat exchangers can be included in gas furnaces, although one having skill in the art will recognize that the disclosed technology can be applicable to multiple scenarios and applications.

For example, a hand sanitizing device according to the disclosed technology can include one or more storage chambers for storing salt, water, and/or additives and a reaction chamber configured to receive one or more substances from one or more of the storage chambers. As will be described more fully herein, the hand sanitizing device can electrolyze the solution in the reaction chamber, the electrolyzed solution can be dispensed for use by the user. that can draw fluid out of the holding tanks and dispense fluid to a user. Alternatively, or additionally, the hand sanitizing device can include a storage tank to store the hand sanitizer before dispensing.

Although certain examples of the disclosure are explained in detail, it is to be understood that other examples and applications are contemplated. Accordingly, it is not intended that the disclosure is limited in its scope to the details of construction and arrangement of components set forth in the following description or illustrated in the drawings. Other examples of the disclosure are capable of being practiced or carried out in various ways. Also, in describing the disclosed technology, specific terminology will be resorted to for the sake of clarity. It is intended that each term contemplates its broadest meaning as understood by those skilled in the art and includes all technical equivalents which operate in a similar manner to accomplish a similar purpose.

Herein, the use of terms such as “having,” “has,” “including,” or “includes” are open-ended and are intended to have the same meaning as terms such as “comprising” or “comprises” and not preclude the presence of other structure, material, or acts. Similarly, though the use of terms such as “can” or “may” are intended to be open-ended and to reflect that structure, material, or acts are not necessary, the failure to use such terms is not intended to reflect that structure, material, or acts are essential. To the extent that structure, material, or acts are presently considered to be essential, they are identified as such.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It is also to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

The components described hereinafter as making up various elements of the disclosure are intended to be illustrative and not restrictive. Many suitable components that would perform the same or similar functions as the components described herein are intended to be embraced within the scope of the disclosure. Such other components not described herein can include, but are not limited to, for example, similar components that are developed after development of the presently disclosed subject matter.

Although the disclosed technology may be described herein with respect to various systems and methods, it is contemplated that embodiments or implementations of the disclosed technology with identical or substantially similar features may alternatively be implemented as methods or systems. For example, any aspects, elements, features, or the like described herein with respect to a method can be equally attributable to a system. As another example, any aspects, elements, features, or the like described herein with respect to a system can be equally attributable to a method.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual rational numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual rational numbers within that range, for example, 1, 2, 2.7, 3, 4, 5, 5.3, and 6. This applies regardless of the breadth of the range.

As used herein, the term “about” should be construed to refer to both of the numbers specified as the endpoint (s) of any range. Any reference to a range should be considered as providing support for any subset within that range. Ranges may be expressed herein as from “about” or “approximately” or “substantially” one particular value and/or to “about” or “approximately” or “substantially” another particular value. When such a range is expressed, other examples can include from the one particular value and/or to the other particular value.

As used herein, the term “on-demand” and/or variations thereof can refer to the performing a task in response to receiving a request for performance of that task, and in the case of a request for a substance or product, the term “on-demand” and/or variations thereof can refer to the generation and dispensing of an amount of the substance in response to receiving a request for the amount of the substance.

Reference will now be made in detail to examples of the disclosed technology, some of which are illustrated in the accompanying drawings. Wherever convenient, the same references numbers will be used throughout the drawings to refer to the same or like parts.

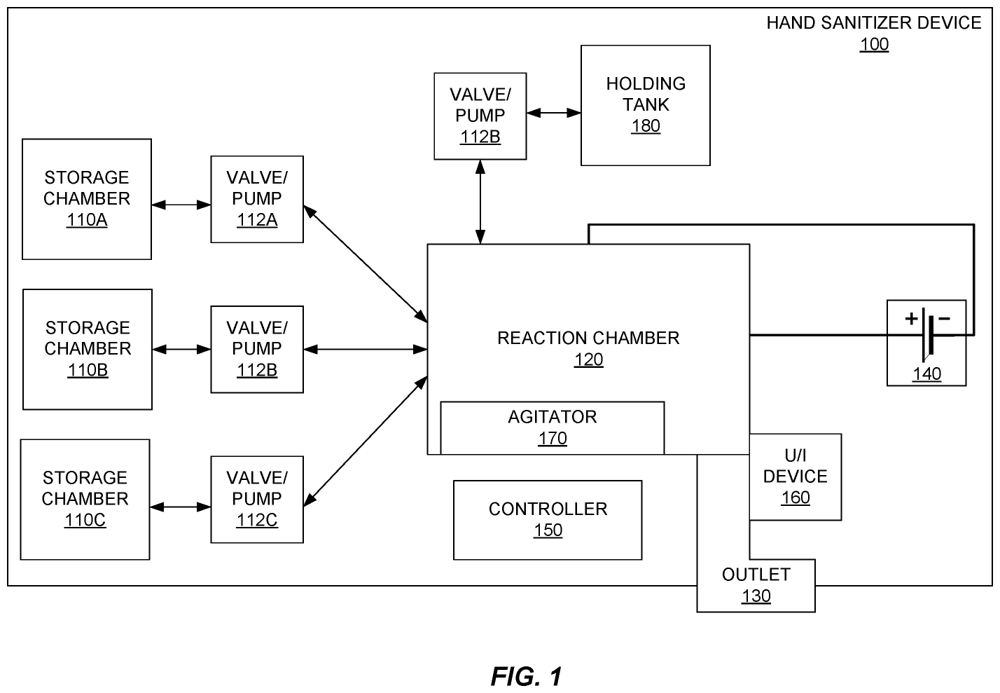

FIG. 1 illustrates a schematic view of a hand sanitizer device 100, which can include one or more storage chambers 110A, B (collectively 110), a reaction chamber 120, and/or an outlet 130 through which an electrolyzed saltwater product can be discharged. The storage chambers 110 can be in selective fluid communication with the reaction chamber 120. The reaction chamber 120 can be connected to an electricity source140 that is configured to provide a current through the reaction chamber 120 to electrolyze the contents in the reaction chamber 120 and create an electrolyzed saltwater product. The electricity source 140 can be a battery, an outlet connected to an electrical grid (e.g., a 120V outlet), a solar cell, or any source of electricity.

As shown in FIG. 1, the hand sanitizer device 100 can include a third storage chamber 110C. The third storage chamber 110C can be in selective fluid communication with the reaction chamber 120. The third storage chamber 110C can store an additive, which can be provided into reaction chamber 120 to be incorporated into the electrolyzed saltwater product. The additive(s) can be or include a moisturizer, a surfactant, a vinegar, an emulsifier, a gel, and the like. The additive(s) can also include catalysts, reagents, and the like that can help quicken the electrolysis reaction in the reaction chamber 120. The additive can provide benefits to the hand sanitizer and/or make the hand sanitizer more pleasant to use. The third storage chamber 110C can also be configured to contain more than one additive, containing as many additives as desired to be added to the hand sanitizer. The third storage chamber 110C can be configured to mix the additive into the reaction chamber 120 before the current is applied or after the current is applied but prior to dispensing the hand sanitizer.

The hand sanitizer device 100 can include one or more valves, pumps, or other devices for releasing, expelling, or otherwise transporting one or more substances (e.g., water, one or more reagents, electrolyzed saltwater product). For example, the hand sanitizer device 100 can include a valve 112A configured to selectively release water (e.g., from an external water source, from a first storage chamber 110A) into the reaction chamber 120 and can include a valve 112B configured to selectively release a salt (e.g., from a second storage chamber 110B) into the reaction chamber 120. The hand sanitizer device can include a valve 112 corresponding to each storage chamber 110 such that the hand sanitizer device 100 can selectively release a substance from the storage chamber 110 and introduce the substance into the reaction chamber 120. Once all substances are introduced into the reaction chamber 120, the mixture of water, salt and/or other reagents can be electrolyzed as described herein. Alternatively, or additionally, the hand sanitizer device 100 can include one or more pumps to transport the water, salt, and/or other reagents. The hand sanitizer device 100 can include a single pump configured to move at least some of the water, salt, and/or other reagents into the reaction chamber 120. Alternatively, the hand sanitizer device 100 can include multiple pumps (e.g., a separate pump for each storage chamber 110).

A given valve, pump, and/or other device can be configured to selectively release, expel, and/or otherwise transport a predetermined amount of a corresponding substances. For example, a first valve or first pump 112 can be configured to release or transport a predetermined amount of water or solution into the reaction chamber, a second valve or second pump 112 can be configured to release or transport a predetermined amount of salt into the reaction chamber, and/or a third valve or third pump 112 can be configured to release or transport a predetermined amount of an additive into the reaction chamber 120. Each valve and/or pump 112 can be controlled by the controller 150, as described more fully herein.

The hand sanitizer device 100 can optionally include an agitator 170 disposed within the reaction tank, which can help form a substantially mixed mixture within the reaction chamber 120. The agitator 170 can be used to agitate or mix the solution or mixture within the reaction chamber 120 before, during, and/or after electrolysis is performed. The agitator 170 can include an auger, an impeller, a magnetic agitator, a mixing paddle, one or more jets, or the like.

The hand sanitizer device 100 can include a controller 150 configured to control and/or communicate with one or more of the various components described herein (e.g., one or more valves, one or more pumps, one or more electricity sources 140, one or more sensors). The controller 150 can include one or more processors and memory. The memory can have instructions stored thereon that, when executed by the one or more processors, cause the controller 150 to output instructions to one or more components of the hand sanitizer device 100. That is, the controller 150 can perform one or more of the methods described herein, and/or the controller 150 can output instructions that, when executed by one or more components, cause the hand sanitizer device 10 to various perform one or more of the methods described herein.

The hand sanitizer device 100 can include a user input device 170. The user input (U/I) device 170 can be any button, switch, or interface configured to receive input for a user. The input can correspond to a request for an amount of electrolyzed saltwater product for performing a hand disinfecting action (e.g., a hand wash with the electrolyzed saltwater product). Alternatively, or additionally, the U/I device 170 can include a sensor configured to detect a user. For example, the U/I device 170 can include a proximity sensor, an infrared sensor, or the like. The U/I device 170 can thus be configured to detect when a user or a part of a user (e.g., a user's hand) comes within a predetermined distance from the U/I device 170. The U/I device 170 can be in electrical communication with the controller 150 and can be configured to transmit signals to the controller 150. The signals can be indicative of a request for an amount of electrolyzed saltwater product for performing a hand disinfecting action (e.g., detecting a user or a part of a user within the predetermined distance from the U/I device 170).

The various components described above in the hand sanitizer device 100 can be included in a single housing. The housing can have an opening through which the outlet 130 can dispense hand sanitizer. The housing can be mountable (e.g., on a wall, on a dedicated stand) or free-standing. The housing can also be disassembled such that the various components of the hand sanitizer device 100 can be accessed (e.g., for replacement or maintenance). The housing can also have internal partitions to separate the components of the hand sanitizer device 100. For example, the controller 150 and other electrical components can have their own compartment within the housing separate from the storage chamber 110 and the reactive chamber 120. Additionally, the housing can contain the U/I device 170, allowing a user to easily interact with the U/I device from outside of the housing.

Alternatively, or additionally, some of the components of the hand sanitizer device 100 can be contained external from the housing. For instance, the housing can contain the storage chamber 110 and the reactive chamber 120 while the controller 150 and the electricity source 140 remain separate from, but connected to, the housing. In such a manner, certain electrical components of the hand sanitizer device 100 can be kept distant from the chemical components to prevent potential safety issues.

The hand sanitizer device 100 can be configured to receive water from an external water source. Alternatively, the hand sanitizer device 100 can be configured to store water in one of the storage chambers 110. The hand sanitizer device 100 can be configured to store a salt in another storage chamber 110. Alternatively, the hand sanitizer device 100 can be configured to store saltwater (i.e., a water-based solution having at least one salt dissolved therein) in a storage chamber 110.

The salt can be any water-soluble compound, such as any alkali or alkaline salt. For example, the salt can be sodium chloride. The salt can further be any compound that can form an electrolyte when dissolved in water. In such a manner, when the salt of the second storage chamber 110B is mixed with the water of the first storage chamber 110A in the reaction chamber 120, an electrolytic cell can be formed.

As will be appreciated, dissolving a salt in water can create an anion and a cation. The concentration of the anion can be controlled to create a particular product as desired. For example, the salt used by the hand sanitizing device 100 can be or include sodium chloride, and the hand sanitizing device 100 can be configured to control the concentration of chloride ions in the reaction chamber 120 to create an electrolyzed saltwater product comprising hypochlorous acid. To produce an electrolyzed saltwater product having a sufficient disinfectant aspect (e.g., for use against COVID-19), the hand sanitizer device 100 can be configured to provide a concentration of chloride ions that is greater than or equal to approximately 50 ppm and/or is less than or equal to approximately 200 parts per million (ppm).

The hand sanitizer device 100 can be configured to apply a current from the electricity source 140 to the reaction chamber 120 (e.g., via an electrolytic cell 220, as described more fully herein) when a combination or mixture of certain substances (e.g., one or more of the substances stored in containing the contents from the first storage chamber 110A and the second storage chamber 110B, an electrolyzed saltwater product can be formed. The electrolyzed saltwater product can then be dispensed through the outlet 130.Alternatively or additionally, the electrolyzed saltwater product can be moved to a holding tank 180. The holding tank 180 can be separate from, but in fluid communication with, the reaction chamber 120. Alternatively, the reaction chamber 120 can function as the holding tank 180 when the electric potential is not applying a current to the reaction chamber 120.

The product can be created from the electrolysis of the salt and the water. For example, the product can be hypochlorous acid. As described above, the outlet 130 can be attached to the outside of a housing to dispense the product to the user. The product can be dispensed through the outlet 130 in a variety of forms. The outlet 130 can comprise an atomizer to dispense the product in a mist form. As would be appreciated, dispensing the product in mist form can conserve the volume of product while eliminating the need for a drain or catcher to catch excess product that does not make it on to the user's hands. In other examples, the outlet 130 can dispense the product in a liquid form. The outlet 130 can also be disposed within a recess in the housing into which a user can insert his or her hands. In such a manner, the user can insert his or hands into the recess, and the hand sanitizer device 100 can dispense hand sanitizer onto the user's hands without contact between the hand sanitizer device 100 and the user (e.g., rather than dispensing a quantity into the user's hands and requiring the user to spread the hand sanitizer). The recess can also be configured to collect any overspray from the outlet 130 (e.g., by a collection drain located in the recess (e.g., at a bottom surface of the recess). The outlet 130 can also have an ultraviolet (UV) light source to help disinfect a user's hands with UV light. The UV light source can be configured to provide UV light in the UV-C band. The UV light source can also be disposed within the recess in the housing such that the user's hands are fully exposed to UV light.

The outlet 130 can be a single aperture to dispense the hand sanitizer to a single point, or the outlet 130 can comprise multiple apertures to dispense the hand sanitizer to a predetermined area. For example, as described above when the outlet 130 is disposed within a recess, the outlet 130 can have multiple apertures within the recess. In such a manner, the hand sanitizer can be dispensed from various angles to provide a larger area of coverage. In other examples, however, the outlet 130 can have just a single aperture to dispense an amount of hand sanitizer, allowing users to spread the hand sanitizer themselves.

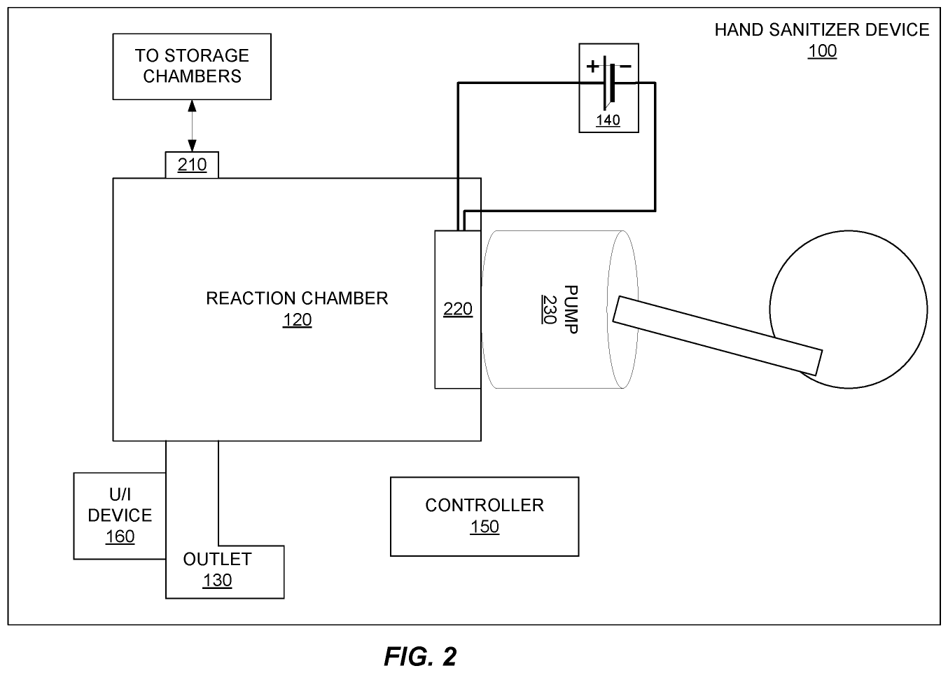

FIG. 2 illustrates an example of a reaction chamber 120. As shown, the reaction chamber 120 can have an inlet 210 connected to any number of holding tanks, such as the first storage chamber 110A and/or the second storage chamber 110B. The reaction chamber 120 can also be connected to the outlet 130 configured to dispense a quantity of the product. The reaction chamber 120 can also contain an electrolytic cell 220. The electrolytic cell 220 can be in electrical communication with the electricity source 140, and the electrolytic cell 220 can be configured to apply a current to the reaction chamber 120. Alternatively or additionally, as described above, the electricity source 140 can be connected to the reaction chamber to create the electrolytic cell 220. That is, an electrolytic cell can be located within the chamber and/or the chamber can act as an electrolytic cell itself.

The electrolytic cell 220 can include electrodes, such as a cathode and an anode. The electrodes can comprise any conductor or semiconductor, such as a metal. Suitable examples of a metal can include, but are not limited to, graphite, silver, copper, gold, aluminum, calcium, tungsten, zinc, nickel, lithium, iron, platinum, tin, gallium, niobium, steel, carbon steel, lead, titanium, electrical steel, manganin, constantan, stainless steel, mercury, manganese, amorphous carbon, germanium and the like. The electrodes can be in the form of a mesh, a plate, a disc, and the like capable of providing a reactive surface.

The reaction chamber 120 can include a pump 230, which can be any pressure differential-creating device or other device configured to transport or expel the electrolyzed saltwater product. For example, the pump 230 can be configured to create a pressure differential such that the electrolyzed saltwater product, or any other contents of the reaction chamber 120, can be forced out of the outlet 130. The pump 230 can also be used to draw the reagents out of the storage chambers (e.g., the first and the second storage chambers 110). The pump 230 can work in conjunction with, or in place of, the valves 112. The pump 230 can be or include a vacuum pump, a self-priming pump, a piston pump, or the like. In FIG. 2, the pump 230 is shown as a piston connected to a flywheel to illustrate an alternative pressure-differential creating device. The pump 230 can be attached to an exterior portion of the hand sanitizer device 100, rather than inside the reaction chamber 120. Any harsh environments within the reaction chamber 120 may be detrimental to the pump 230, thus reducing the operating lifetime and increasing maintenance costs.

The reaction chamber 120 can comprise one or more sensors for measuring properties of the components within the reaction chamber 120. For example, the reaction chamber 120 can include a temperature sensor, a pH sensor, and/or a voltmeter. For example, the controller 150 can receive inputs from the aforementioned sensors and make determinations to ensure that the reaction in the reaction chamber 120 is being carried out properly. For instance, if the pH within the reaction chamber 120 deviates too far from a predetermined value, the controller 150 can instruct one of the storage chambers 110 (e.g., the first or the second storage chambers) to add an additional quantity of a reagent to stabilize the reaction.

The reaction chamber 120 can also include a heating element to facilitate and/or speed up the reaction. The controller 150 can utilize a temperature sensor (as described above) to monitor the temperature within the reaction chamber 120, and the controller 150 can provide instructions to the heating element if there is too much or too little heat detected by the temperature sensor. The reaction chamber 120 can be maintained at a temperature of 77° C. or less.

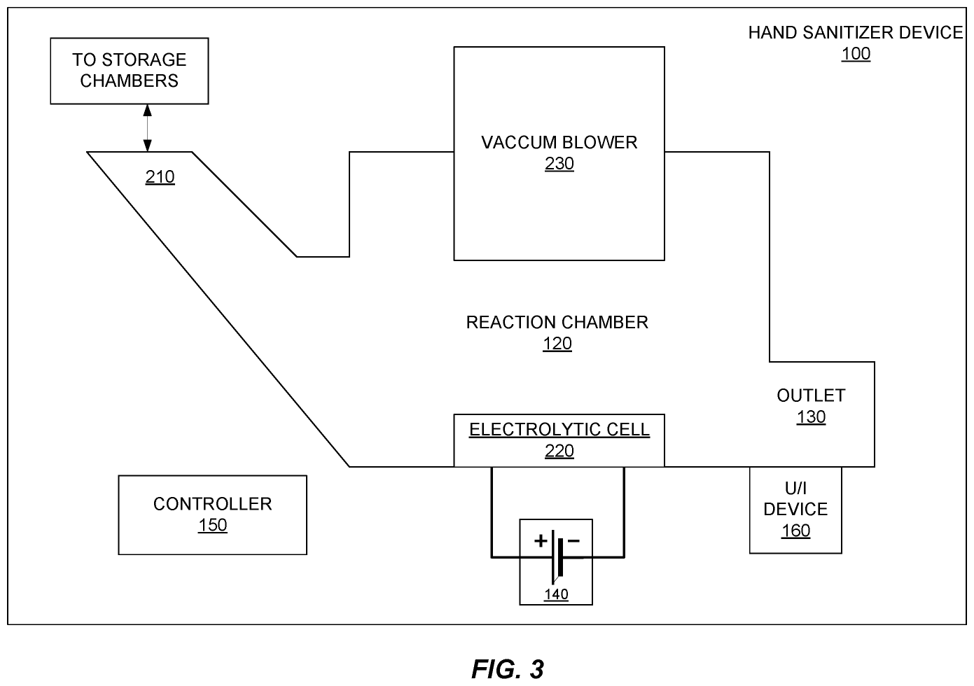

FIG. 3 illustrates another example of the reaction chamber 120 and the pump 230. In FIG. 3, the pump 230 is shown as a vacuum blower pump. The reaction chamber 120 can still comprise an inlet 210 connected to any number of storage chambers 110, such as the first storage chamber 110A and/or the second storage chamber 110B. The reaction chamber can also be connected to the outlet 130 configured to dispense a quantity of the product. The reaction chamber 120 can also contain an electrolytic cell 220. The electrolytic cell 220 can be in electrical communication with the electricity source 140, and the electrolytic cell 220 can be configured to apply a current to the reaction chamber 120.

The reaction chamber 120 can be connected to the holding tank 180 and the pump 230 can be contained within the holding tank 180 rather than in the reaction chamber 120. Additionally, the reaction chamber 120 can have its own pump, such as the valves 112, configured to move the product from the reaction chamber 120 to the holding tank 180.

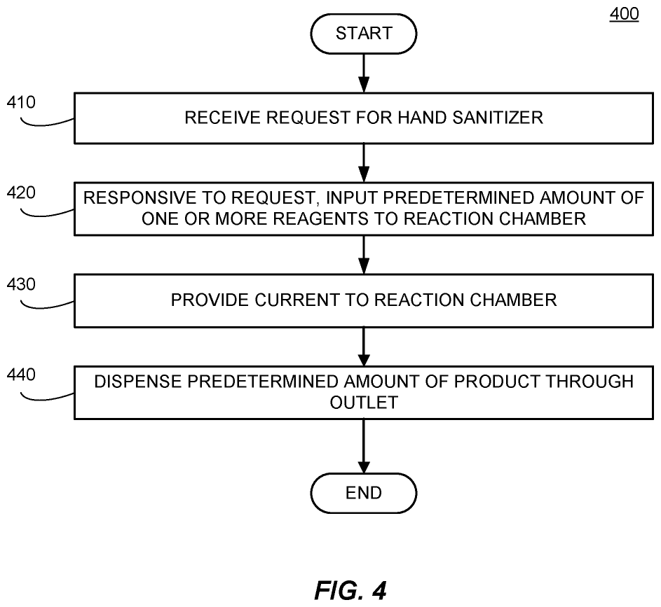

FIG. 4 illustrates a method 400 of dispensing hand sanitizer according to the present disclosure. While the method 400 is described with reference to the controller 150, it is understood that the method 400 can be implemented by other similar systems or components.

In block 410, the method 400 can include receiving a request for hand sanitizer (e.g., from the U/I device 170). The request can be made, for example, by a user pushing a button or by a motion sensor detecting the presence of the user and transmitting motion data to the controller 150.

In block 420, the method 400 can include, in response to receiving the request, instructing one or more valves or pumps (e.g., valves 112) to introduce into the reaction chamber 120 a predetermined amount for each of one or more reagents. The one or more reagents can be combined in the reaction chamber 120 in any desired ratio. For example, the controller 150 can instruct the valve 112A to dispense a predetermined amount of water, and the controller 150 can instruct the valve 112B to dispense a predetermined amount of salt. The controller 150 can also instruct the third storage chamber 110C to dispense an additive into the reaction chamber 120. The method 400 can then proceed on to block 430.

In block 430, the method 500 can include instructing the electricity source 140 to provide a current to the reaction chamber 120 (e.g., via the electrolytic cell 220). The applied current can cause an electrolysis reaction to occur in the reaction chamber 120, thereby causing the one or more reagents to form a product. An additive can be added to the reaction chamber 120 from a third reagent tank 110C, and the additive can be added before, during, and/or after the electrolysis reaction. The electricity source 140 can be configured to apply a sufficient current to the reaction chamber 120. The sufficient current can be from approximately 0.5 A to approximately 2 A corresponding to a 12 V electricity source 140.

The current can be applied for a predetermined duration. The predetermined duration can be a sufficient time period for an electrolysis reaction to sufficiently occur in the reaction chamber 120. For example, the current can be applied to the reaction chamber 120 for 0.1 seconds or greater (e.g., 0.2 seconds or greater, 0.3 seconds or greater, 0.4 seconds or greater, 0.5 seconds or greater, 0.6 seconds or greater, 0.7 seconds or greater, 0.8 seconds or greater, 0.9 seconds or greater, 1 second or greater, 1.1 seconds or greater, 1.2 seconds or greater, 1.3 seconds or greater, 1.4 seconds or greater, 1.5 seconds or greater, 1.6 seconds or greater, 1.7 seconds or greater, 1.8 seconds or greater, 1.9 seconds or greater, or 2.0 seconds or greater), based on a current of 1 ampere and a 12 V electricity source 140.

The current can be applied to the reaction chamber 120 for 2 seconds or less (e.g., 0.2 seconds or less, 0.3 seconds or less, 0.4 seconds or less, 0.5 seconds or less, 0.6 seconds or less, 0.7 seconds or less, 0.8 seconds or less, 0.9 seconds or less, 1 second or less, 1.1 seconds or less, 1.2 seconds or less, 1.3 seconds or less, 1.4 seconds or less, 1.5 seconds or less, 1.6 seconds or less, 1.7 seconds or less, 1.8 seconds or less, 1.9 seconds or less, or 2.0 seconds or greater), based on a current of 1 ampere and a 12 V electricity source 140.

The current can be applied to the reaction chamber 120 from 0.1 seconds to 2 seconds (e.g., from 0.2 seconds to 1.9 seconds, from 0.3 seconds to 1.8 seconds, from 0.4 seconds to 1.7 seconds, from 0.5 seconds to 1.6 seconds, from 0.5 seconds to 1.5 seconds, or from 1 second to 2 seconds). The method 400 can then proceed on to block 440.

In block 440, the method 400 can include instructing the pump 230 to dispense a predetermined amount of the product through the outlet 130. The created pressure differential can cause the product to be expelled and/or forced out of the outlet 130. The pump 230 can be configured such that any predetermined quantity can be dispensed at a time. The outlet 130 can also have one or more apertures to dispense the product to a larger surface area, or the outlet 130 can have a single aperture to dispense the product to a single point.

It is understood that the method 400 can be performed on-demand. That is to say, from the time when a user requests hand sanitizer and the method 400 begins at block 410, to the time when the hand sanitizer is dispensed at block 550, the passage of time experienced by the user is minimal.

Certain examples and implementations of the disclosed technology are described above with reference to block and flow diagrams of systems and methods according to examples or implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, may be repeated, or may not necessarily need to be performed at all, according to some examples or implementations of the disclosed technology. That is, the disclosed technology includes the performance of some, or all steps of the methods and processes described herein in conjunction with the performance of additional steps not expressly discussed herein. Further, the present disclosure contemplates methods and processes in which some, but not all, steps described herein are performed.

While the present disclosure has been described in connection with a plurality of example aspects, as illustrated in the various figures and discussed above, it is understood that other similar aspects can be used, or modifications and additions can be made to the described aspects for performing the same function of the present disclosure without deviating therefrom. For example, in various aspects of the disclosure, methods and compositions were described according to aspects of the presently disclosed subject matter. However, other equivalent methods or composition to these described aspects are also contemplated by the teachings herein. Therefore, the present disclosure should not be limited to any single aspect, but rather construed in breadth and scope in accordance with the appended claims.

### Example Use Case

The following examples describe examples of a typical user flow pattern. They are intended solely for explanatory purposes and not limitation.

Salt, water, and an additive in the right proportion (e.g., between approximately 10 grams and approximately 20 grams of salt per 1 liter of water) are pulled into a reaction chamber using a suction mechanism. The reagents within the reaction chamber can be kept at or below 77° F. (e.g., at 74° F.). The salt can be added in an amount such that 50 ppm or greater of free chloride ions can be present in the reaction chamber. The additive can be vinegar. Once all the ingredients are in the reaction chamber, a small current is passed through the electrolytic cell to facilitate the electrolysis process. Since the chamber volume can be small (e.g., have a volume in the range between approximately 3 ml and approximately 10 ml), only a small current and a short amount of time (e.g., a duration in the range between approximately 1 second and approximately 2 seconds) is needed to electrolyze the solution in the reaction chamber and dispense it. Since the current required is small, the unit can be powered by a conventional household socket, battery, or solar cell.

A user can push a button on a wall-mounted hand sanitizer dispenser to request hand sanitizer. The button request can be received and processed by a controller having a memory and a processor. In response, the controller can output instructions for salt and water to be dispensed from two respective storage chambers and into a reaction chamber. The salt can be dispensed in an amount of approximately 3 grams per liter of water dispensed. The controller can also output instructions for vinegar, which can be useful as a disinfection boosting agent, to be dispensed from a third storage chamber. The vinegar can be dispensed in an amount that is between approximately 1 tbsp and approximately 2 tbsp. Then, the controller can instruct an electricity source to apply a current to the reaction chamber (e.g., through an electrolytic cell) for a duration in a range between approximately 2 sections and approximately 3 seconds. This can facilitate an electrolysis reaction to create an electrolyzed saltwater product. The controller can output instructions for the electrolyzed saltwater product to be dispensed through an outlet and to the user in an amount that is approximately 0.02 g (e.g., as an atomized spray or mist). Any remaining product can be transferred from the reaction chamber to a holding chamber for short-term storage.

What is claimed is:

1. A hand sanitizer device comprising:
one or more storage chambers storing at least one of water and salt;
a reaction chamber connected to the one or more storage chambers;
an outlet connected to the reaction chamber;
an electricity source in electrical communication with the reaction chamber; and
a controller having a processor and a memory storing instructions that, when executed by the processor, cause the controller to:
receive a request for an amount of electrolyzed saltwater product for performing a hand disinfecting action;
responsive to receiving the request, output instructions for a predetermined amount of water and a predetermined amount of salt to be introduced into the reaction chamber;
provide a current of electricity from the electricity source and through the reaction chamber, thereby electrolyzing the contents of the reaction chamber to form the amount of electrolyzed saltwater product; and
dispense the amount of electrolyzed saltwater product through the outlet.

2. The device of claim 1, wherein the current of electricity is provided to the reaction chamber for a duration between approximately 1 second and approximately 10 seconds.

3. The device of claim 1, wherein the current of electricity has an amperage between approximately 0.5 amperes and approximately 2 amperes.

4. The device of claim 1, wherein the predetermined amount of salt is between approximately 1 gram and approximately 20 grams per liter of water introduced into the reaction chamber.

5. The device of claim 1, wherein the instructions, when executed by the processor, further cause the controller to:
responsive to receiving the request, introduce a predetermined amount of an additive into the reaction chamber.

6. The device of claim 5, wherein the additive is selected from the group consisting of: a moisturizer, a surfactant, a vinegar, an emulsifier, and a gel.

7. The device of claim 1, further comprising a housing inside of which the one or more storage chambers, the reaction chamber, and the outlet are at least partially disposed.

8. The device of claim 7, wherein the housing comprises a recess and the outlet is disposed within or proximate the recess.

9. The device of claim 8, wherein the outlet comprises one or more apertures configured to dispense the electrolyzed saltwater product in the form of a mist.

10. The device of claim 8, further comprising an ultraviolet (UV) light source configured to output UV light on a user's hand.

11. A hand sanitizer device comprising:
a controller having a processor and a memory storing instructions that, when executed by the processor, cause the hand sanitizer device to:
receive, from a user input device, a request for an amount of electrolyzed saltwater product for performing a hand disinfecting action;
responsive to receiving the request, introducing into a reaction chamber a predetermined amount of water and a predetermined amount of salt;
provide a current of electricity from an electricity source and through the reaction chamber, thereby electrolyzing the contents of the reaction chamber to form the electrolyzed saltwater product; and
dispense the amount of electrolyzed saltwater product through an outlet having one or more apertures.

12. The device of claim 11, wherein the current of electricity is provided to the reaction chamber for a duration between approximately 1 second to approximately 10 seconds.

13. The device of claim 11, wherein the current of electricity has an amperage between approximately X amperes and Y amperes.

14. The device of claim 11, wherein the predetermined amount of salt is between approximately 1 gram and 20 grams per liter of water introduced into the reaction chamber.

15. The device of claim 11, wherein the instructions, when executed by the processor, further cause the controller to:
responsive to receiving the request, introduce a predetermined amount of an additive into the reaction chamber.

16. The device of claim 15, wherein the additive is selected from the group consisting of: a moisturizer, a surfactant, a vinegar, an emulsifier, and a gel.

17. A method comprising:
receiving, from a user input device, a request for an amount of electrolyzed saltwater product for performing a hand disinfecting action;
responsive to receiving the request, introducing a predetermined amount of water and a predetermined amount of salt into a reaction chamber;
providing a current of electricity from an electricity source and through the reaction chamber, thereby electrolyzing the contents of the reaction chamber to form the electrolyzed saltwater product; and
dispensing the amount of electrolyzed saltwater product through an outlet having one or more apertures.

18. The device of claim 17, wherein the current of electricity has an amperage between approximately X amperes to Y amperes.

19. The device of claim 17, wherein the instructions, when executed by the processor, further cause the controller to:
responsive to receiving the request, introduce a predetermined amount of an additive to the reaction chamber.

20. The device of claim 19, wherein the additive is selected from the group consisting of: a moisturizer, a surfactant, a vinegar, an emulsifier, and a gel.
